(19) United States
(12) Patent Application Publication
(10) Pub. No.: US 2017/0239466 A1
(43) Pub. Date: Aug. 24, 2017

(54) IONTOPHORETIC SOFT CAPSULE

(71) Applicant: L'OREAL, Paris (FR)

(72) Inventors: Jennyfer CAZARES DELGADILLO, Paris (FR); Dominique BORDEAUX, Soisy Sur Seine (FR)

(21) Appl. No.: 15/517,590

(22) PCT Filed: Sep. 24, 2015
(86) PCT No.: PCT/EP2015/071994

§ 371 Date: Apr. 7, 2017

(30) Foreign Application Priority Data
Oct. 7, 2014 (FR) 1459604

(51) Int. Cl. A61N 1/30; A61Q 19/00; A61K 8/02; A61L 2/20; A45D 40/26; A61L 2/08; A61L 2/10; A61N 1/04; A61N 1/32; A45D 34/04; A61K 8/11; A61L 2/04

(52) U.S. Cl. CPC A61N 1/303; A61K 8/11; A61Q 19/00; A61K 8/0204; A61L 2/206; A61L 2/04; A61L 2/081; A61L 2/10; A61L 2/085; A61N 1/044; A61N 1/325; A61N 1/328; A45D 34/04; A45D 40/26; A61K 2800/83; A61K 2800/87; A61L 2202/23

(57) ABSTRACT

The present invention relates to a device for applying a cosmetic composition (P), comprising: —a capsule (1) that delimits a housing (10), —an application member (6) positioned in the housing (10) and impregnated with a cosmetic composition (P), —an electric exciter (29) comprising at least two electrodes (4, 5) in contact with the application member (6) and making it possible to circulate an electric current between these two electrodes (4, 5).

One subject of the present invention is a device for applying a composition, in particular a cosmetic composition, including a care composition.

The term “cosmetic product” is understood, for the purposes of the present invention, to mean a composition as defined in Directive 93/35/EEC dated 14 Jun. 1993 modifying Directive 76/768/EEC.

Known from patent application US 2003/0129016 is a passive device comprising a container that contains the product to be applied and a porous structure that defines a surface used for the application, supplied with the product.

However, the amount of molecules administered via the passive route remains low. In order to improve this aspect, active patches or masks have been developed that use electrodes connected to a power source.

It is thus known to treat human keratin materials using iontophoresis devices (J. Singh, K. S. Bhatia, Topical iontophoretic drug delivery: pathways, principles, factors and skin irritation, Med. Res. Rev., vol. 16, no. 3, 285-296, 1996).

Iontophoresis allows the diffusion of active agents through the skin by virtue of electrical stimulation in a non-invasive manner. The current applied may be adjustable in terms of intensity and direction (anodal or cathodal). The transcutaneous diffusion of the molecules via iontophoresis is based on two principles, namely electrorepulsion and electroosmosis.

Electrorepulsion is the migration of an ionized molecule by repulsion of charges of the same sign. Thus, if a substance has a positive charge, it will diffuse through the skin at the anode (+).

Electroosmosis is the migration of a molecule, even a non-ionized molecule, by entrainment associated with the flow of water from the anode to the cathode during iontophoresis. The migration is due in particular to the negative charge of the skin. Under the effect of a current, the water or a solvent entrains dissolved substances as it migrates.

U.S. Pat. No. 7,597,495 describes a device for applying a composition, comprising:
an application member having a surface suitable for being loaded with composition;
an electric exciter comprising at least two electrodes in permanent contact with the application member and making it possible to circulate an electric current between these two electrodes.

The operation of this device is based on the principle of iontophoresis.

However, this device has certain drawbacks:
it comprises a reservoir containing the product and arranged to reload the application member with composition,
the amount of composition delivered varies as a function of the viscosity of the compositions,
the composition may be contaminated in the event of ineffective cleaning carried out by the consumer, the properties of the composition possibly being modified,
the presence of impurities in the composition may reduce the effectiveness of the treatment and also the synergistic effect due to the application of a current,
the formulation is in direct contact with electrodes, which may give rise to a modification of the pH over time, and optionally to skin irritations,
the configuration of the device may result in an ionic competition in the composition, which reduces the effectiveness of the treatment.

The documents FR 2656 223, EP 0 642 808, EP 1 563 868, US 2008/051692 and US 2011/213295 describe devices for applying a cosmetic composition comprising a rigid envelope. The device is difficult to move over the skin. It is not sufficiently effective for confined areas of the body. The result obtained is not homogeneous. The capsule is not crushed by pressure. It does not conform to the sides of the nose or to the cheekbones for example. The performance of these devices can be improved.

The present invention aims in particular to facilitate or reinforce the action of the composition on the region of the body or of the face treated and/or to create effects new to the application.

In particular, the invention proposes a device which:
adapts to the contours of the face and of the body,
does not cause leakage of the cosmetic composition during its use,
regularly delivers the cosmetic composition,
delivers a wide range of textures of compositions,
allows a uniform diffusion of the electric current through the cosmetic composition, in order to avoid local pH variations and skin irritations,
makes it possible to store the cosmetic composition without preservative, in order not to hamper the transport of the active agents through the skin,
is disposable after use to avoid any contamination and to increase the effectiveness of an application,
may be produced as a function of the body area to be treated and of the specificity of the skin,
is effective,
safe,
easy-to-use,
affordable in terms of price,
can be used with various cosmetic compositions.

The subject of the invention is thus, according to a first of its aspects, a device for applying a cosmetic composition, comprising:
a capsule that delimits a housing,
an application member positioned in the housing and impregnated with a cosmetic composition,
an electric exciter comprising at least two electrodes in contact with the application member and making it possible to circulate an electric current between these two electrodes.

Preferably, the capsule is made of soft material.

The term “contact” is understood to mean the fact that the electrodes and the application member are kept in contact at least when the device is operational. This does not rule out that the application member, where appropriate, may be separated from the electrodes, in particular in order to be replaced by another, between two uses of the device. Preferably, the contact is permanent during the operation of the device.

The invention offers numerous advantages.

Firstly, the application member is hygienic.

Furthermore, the application device according to the invention may be handled relatively easily.

The circulation of the current between the electrodes may for example promote the opening and dilation of the pores of the skin and facilitate the action and/or the penetration of at least one active agent of the composition in the skin.

The invention may also make it possible, where appropriate, to activate blood microcirculation, to improve the muscle tone or the healing capacity of the skin.

Application Member

An “application member” refers to a material, in particular a porous or fibrous material, capable of retaining a cosmetic composition by impregnation. It comprises an application surface intended to come into contact with the body surface.

The application member is in particular an absorbent material.

The expression “impregnated application member” denotes an application member in which a cosmetic composition is distributed in a diffuse manner.

The application member comprises an application surface intended to come into contact with the body surface.

According to one embodiment of the device of the invention, the application member is compressible.

In one example of use of the invention, the application member is electrically conductive when dry.

Thus, electric current may circulate between the two electrodes independently of whether the product to be applied is electrically conductive or not. This may make it possible, where appropriate, to locally heat the application member.

The application member may comprise, for example, a material that is intrinsically not very conductive, for example a plastic, containing an electrically conductive filler. This filler may in particular contain electrically conductive particles, for example metal particles, selected in particular from the following list: particles of copper, of aluminium, of silver and of graphite.

In another example of use of the invention, the application member is electrically insulating when dry.

In order to increase the intensity of the electric current circulating between the electrodes, the application member may be electrically conductive when dry and the product may also be electrically conductive.

In one example of use of the invention, the application member is porous or fibrous. The application member may in particular comprise at least one of the members selected from the following list: a nonwoven, a felt, paper, a foam, a sponge, a sintered product, a porous ceramic, a biocellulose substrate, an alginate foam, a polysaccharide, a hydrogel, etc.

The application member may be capable of retaining the composition by capillary action and may comprise, for example, a tuft of pile or a soft part, in particular made of silicone or elastomer, suitable for retaining the composition by capillary action.

The application member may comprise a multilayer structure with, for example, at least one conductive layer sandwiched between two other layers. The application member may comprise, for example, successively from the outside inwards, an outer layer made of felt, a conductive layer, for example made of metal, in particular made of aluminium, and a foam layer, it being possible for at least one of the electrodes to be connected to the conductive layer.

The application member may optionally be hydrophilic.

The application member may also comprise magnetic particles in order to combine the effects of the current and of a magnetic field.

Nonwoven

For the purposes of the present invention, the expression “nonwoven” is understood to mean a substrate comprising fibres, in which substrate the individual fibres or the filaments are arranged in a disordered manner in a structure in the form of a sheet and which are neither woven nor knitted. The fibres of the nonwoven are generally bonded together, either under the effect of a mechanical action (for example needle punching, air jet, water jet, etc.), or under the effect of a thermal action, or by addition of a binder.

Such a nonwoven is, for example, defined by standard ISO 9092 as a web or sheet of directionally or randomly orientated fibres, bonded by friction and/or cohesion and/or adhesion, excluding paper and products obtained by weaving, knitting, tufting or stitching incorporating binding yarns or filaments.

Advantageously, the weight percentage of hot-melt fibres contained in the nonwoven is greater than 0.5% and is less than or equal to 100%, advantageously between 5% and 80%.

The hot-melt fibres are for example polyolefin fibres, such as polyethylene (PE), polypropylene (PP) or polyethylene terephthalate (PET) fibres or acrylic fibres such as polymethyl methacrylate (PMMA) fibres, polyurethane fibres or fibres of the following thermoplastics: polyvinyl chloride (PVC), styrene polymers (for example polystyrene PS, expandable polystyrene EPS, acrylonitrile-butadiene-styrene terpolymer ABS, styrene-acrylonitrile copolymer SAN, styrene-butadiene copolymer SB), polyamides (PA), polycarbonates (PC), saturated polyesters (for example polyethylene terephthalate glycol PET, polybutylene terephthalate glycol PBT), polyacetals (for example polyoxymethylene POM, trioxane-ethylene oxide copolymer), polyvinyl alcohol (PVA), or else fluoropolymers (for example polytetrafluoroethylene PTFE, polyvinylidene fluoride PVDF, polychlorotrifluoroethylene PCTFE).

The other fibres constituting the nonwoven are for example synthetic fibres derived from petroleum derivatives, natural fibres derived from plants or animals, and/or modified natural fibres, derived for example from treatment or regeneration processes in order to form fibres.

The nonwoven may be formed from one or more consolidated fibre webs.

Felt

The term “felt” signifies here a fibrous structure comprising filaments entangled in all directions. Such a structure may be relatively compact.

A major portion of the fibres may have branchings.

Paper

The term “paper” is understood to mean a material made with vegetable fibres reduced to pulp, spread out and dried in order to form a thin sheet.

Foam

The foam is a medium consisting of a solid or liquid material intimately mixed with gas.

The foam is preferably a hydrophilic foam. The term “hydrophilic” is understood to mean a foam that absorbs water. Preferably, the foam used according to the invention has a maximum water absorption capacity of 0.4 to 3.5 g/cm³ and a water retention capacity of 0.07 to 2 g/cm³.

The foam preferably comprises open or semi-open cells.

Furthermore, this foam is advantageously elastic. The term “elastic” is understood to mean a foam that has an elasticity ranging from 10% to 500% elongation, preferably from 50% to 150% elongation.

The foam is preferably made of polyurethane foam, as described for example in document DE 10327707. These are open-cell polyurethane foams, preferably having fine pores. The polyurethane foam may be obtained according to the conventional techniques for preparing polyurethanes, for example by preparation of a polyurethane prepolymer having free isocyanate groups, this prepolymer being obtained from a diisocyanate and a polyol or a polyester or a polyether polyol, then hydrolysis of the prepolymer in contact with water or reaction with an amino compound, in the presence of appropriate catalysts. Use may also be made of any other preparation process known to a person skilled in the art.

The foam may also be made of NBR (nitrile butylene rubber), SBR (styrene butadiene rubber), NR (rubber), PE (polyethylene), EVA (ethylene vinyl acetate) latex, silicone, isoprene, styrene, SEBS (styrene ethylene butylene), PVA (polyvinyl alcohol), polyether, polyester, polyvinyl chloride, this list not being limiting.

Sponge

The sponge is a material of animal or synthetic origin. Its porous structure (it is an open solid foam) gives it a high absorption capacity, approximately twenty-two times its dry weight.

The main sponges of natural origin are sea sponges or vegetable sponges (luffa).

The main sponges of artificial origin originate from the transformation of a natural polymer such as cellulose: cellulose sponge.

As a synthetic sponge, use is preferably made of sponges made of polyurethane foam.

Sintered Product

A sintered product is obtained by sintering. Sintering is a process for manufacturing parts that consists in heating a powder without melting it. Under the effect of the heat, the grains weld together, which forms the cohesion of the part.

Sintering is the consolidation, via the action of heat, of a more or less compact granular agglomerate, with or without melting of one or more of its constituents.

Porous Ceramic

Advantageously, the porous ceramics are selected from:

(i) oxides:
products based on silica (SiO₂),
products with 30% to 100% alumina (Al₂O₃):
   aluminosilicates (clay),
   alumina-zirconia-silica (AZS),
   alumina-chromium oxide-silica (ACS),
   alumina-chromium oxide-zirconia-silica (ACZS),
   high content of alumina,
products based on magnesia (MgO),
products derived from zirconium: zirconia (ZrO₂), Y-TZP (yttrium stabilized tetragonal zirconia polycrystals);

(ii) non-oxides:
carbides,
nitrides,
borides.

Biocellulose Substrate

Biocellulose is a fibrous material obtained by aerobic fermentation, in a nutrient aqueous medium, of bacteria of the genus Acetobacter (also known as Gluconacetobacter) (Z. Gromet-Elhanan, S. Hestrin, Synthesis of cellulose by Acetobacter xylinum, J. Bacteriol. 85, 284-292, 1963; U.S. Pat. No. 5,962,277).

Biocellulose may be used in pure form or in a form combined with other types of fibre, for example fibres of natural origin, for example fibres derived from corn, hemp, flax, cotton, jute, kenaf, raffia, ramie, Panama-hat palm, sisal, rush, esparto grass, phormium, coconut, wool, silk, soya, abaca, kumazasa, persimmon, kapok, burdock, cereal or bamboo fibres.

The biocellulose fibres may be loose or bound together and/or bound to other fibres.

More particularly, the biocellulose fibres considered according to the invention are used in an aggregated form, that is to say aggregated with one another, or even with other fibres, in particular as described above.

This type of fibre network has advantageous capabilities in terms of absorption and desorption capacity.

The main bacterial species used is Acetobacter xylinum, although others may also produce biocellulose, for example Acetobacter pasteurianus.

The conditions for culturing this bacterium to produce biocellulose are well known, especially from the publication Factors affecting the yield and properties of bacterial cellulose, A. Krystynowicz et al., J. Indus. Microbiol. Biotech. 29, 189-195, 2002.

Electrodes

At least one of the electrodes, in particular a terminal portion thereof, may be at least partially embedded, in particular permanently, in the application member. It may comprise several heads that make it possible to increase the area of contact with the application member. Such a feature proves advantageous, especially when the electrode in question is metallic, in order to avoid direct contact with the skin.

In one exemplary embodiment of the invention, where the application member comprises a surface intended to come into contact with the area to be treated, at least one of the electrodes does not have any contact with this surface of the application member.

In another exemplary embodiment of the invention, the electrodes may adopt the outer shape of the application member and have a rounded or flat surface, for example.

At least one of the electrodes may, for example, be flush with the outer surface of the application member, this electrode comprising, for example, a rounded head so as not to injure the user.

When the device comprises a gripping member, at least one of the electrodes may be rigidly fastened to the gripping member.

In one exemplary embodiment of the invention, the device comprises a compartment for receiving an electric power source that makes it possible to electrically power the electrodes, for example at least one electric battery, rechargeable battery or capacitor.

In one exemplary embodiment of the invention, during use, the voltage at the terminals of the electrodes is identical to the voltage at the terminals of the electric power source. As a variant, the electric exciter comprises means for connection to a power adapter, in particular a transformer, connected to the power grid.

The voltage at the terminals of the electrodes may be less than 30 volts, being between 1 volt and 9 volts preferably.

The electric exciter may comprise an indicator light that lights up when the current flows between the electrodes, this control possibly comprising, for example, a light-emitting diode, optionally a bi-colour light-emitting diode.

The electric exciter may comprise a member for adjusting the power, for example the voltage and/or the maximum current, in particular a potentiometer or a multi-way contactor.

The electric exciter may comprise a switch, for example with a button, in order to control the electric power of the electrodes and in particular to start or stop the device.

Advantageously, the applicator comprises a compartment for housing an electric power source, this compartment possibly being made in the gripping member for example.

When the device comprises a compartment for housing an electric power source, this compartment may be provided with an elastic return member, positioned in particular in the bottom of the compartment, arranged in order to move the electric power source from a first position in which the electric power source powers the electrodes, to a second position in which the electric power source is disconnected from the electrodes. In order to electrically power the electrodes, the user exerts a force on the electric power source against the return force of the elastic return member and brings the electric power source to the second position. The device may thus be free of a separate switch for the electric power source and its construction is simplified.

The elastic return member may comprise a spring, in particular a helical spring.

The electric current may, where appropriate, be applied sequentially, either manually by manipulating for example a switch of the device, or automatically owing to a suitable electronic circuit.

The electric current may, where appropriate, be applied continuously.

Advantageously, the device is designed so that the current is applied continuously or sequentially.

The Capsule

According to the invention, the term “capsule” is understood in the broad sense. It denotes a container in general such as a capsule, a bag, a sachet, a pouch, an envelope.

Advantageously, the capsule comprises a soft material.

The expression “soft material” is understood in particular to mean a material, the Shore A hardness of which is strictly less than 25, preferably between 1 and 20, and more preferably between 10 and 15.

Preferably, the soft material is:
a sufficiently conductive material so that the current reaches the application surface, and
the Shore A hardness of which is strictly less than 25.

Since a metal foil is not generally able to satisfy all the conditions required for a material for the capsule, in practice other layers, and in particular layers of plastics, are applied to one face or both faces of the metal foil. These layers may be for example layers laminated by extrusion or applied by means of an adhesive agent in the form of thermoplastic films. These laminated materials containing several layers make it possible to provide capsule materials that are particularly suitable for shaping.

For example, a material according to EP 0 863 812 may be chosen. This is a laminated material which comprises a layer of plastic joined by means of an adhesive agent to a layer of oriented polyamide (oPA) itself joined by means of an adhesive agent to a first side of an aluminium foil. The second side of this aluminium foil, opposite the first side, is covered with a layer of PVC by means of an adhesive agent. It is known to use particular polymers, such as polyurethanes, as adhesive agent.

For example, a material according to U.S. Pat. No. 4,284,672 may also be chosen. It comprises an outer layer made of biaxially oriented thermoplastic polymer, a metal intermediate layer, a layer that promotes adhesion between the metal layer and an inner layer which consists of a polymer that can be heat-sealed to itself. The adhesion-promoting layer is in particular a layer of an acrylic acid/ethylene copolymer.

Advantageously, the capsule comprises a multilayer material.

In particular, the capsule comprises:
a) a metal layer;
b) an adhesive agent preferably comprising a copolymer of carboxylic and/or carboxylate units, the carboxylic units possibly being in acid or salt form; and
c) a layer of a non-metallic material, bonded to the metal layer via said adhesive agent.
The material of the capsule may comprise a conductive polymer.
Preferably, the material of the capsule is obtained by mixing a metal and a polymer by means of a beam of ions.
More advantageously, the beam of ions is a beam of metal ions.
More advantageously still:
   the metal is selected from Al, Sn, Ni, Cu, Fe, Zn, Co, Zr;
   the metal ions are selected from Al³⁺, Sn²⁺, Ni⁺, Cu²⁺, Fe²⁺, Zn²⁺, Co⁺, Zr²⁺;
   the polymer is selected from polyethers, polyesters, polyurethanes, polyolefins or derivatives thereof.
In particular, use is made, as material of the capsule, of a material obtained by mixing a tin film and a polyether ether ketone (PEEK) substrate, the mixing being carried out using an Sn⁺ ion beam. The conductivity of this material may vary, depending on the operating conditions, between 0 and superconductivity.
Such a material is described in “A Tunable Metal Organic Resistance Thermometer”, Dr. Andrew P. Stephenson, Prof. Adam P. Micolich, Dr. Kwan H. Lee, Prof. Paul Meredith and Prof. Ben J. Powell Chem Phys Chem, Volume 12, Issue 1, pages 116-121, Jan. 17, 2011.

The capsule is preferably a removable and disposable capsule.

It is in this case a refill.

It may be disposed of after use.

Preferably, the capsule is attached to the device by snap-fastening, click-fastening, bonding, force-fitting, screwing or magnetization.

The capsule advantageously has a tubular body.

The shape of the capsule may be adapted to the area of the body to be treated.

It may have a cross section that is circular, polygonal, triangular, elliptical, oval, ovoid, cruciform, elongated or flattened.

For example, a triangular cross section may be suitable for the sides of the nose.

A circular cross section may be suitable for the cheekbones.

Preferably, the capsule is sealed by a lid.

The body may be produced with a flange at one end and may have, for example, a shape that is generally rotationally symmetrical about an axis X.

The capsule may be sealed at one end by a bottom wall and at the other end by a sealing wall, in particular a lid.

The sealing wall may comprise, for example, a film which is heat-sealed to the body. This film comprises, for example, at least one layer of soft material.

The sealing wall may also be bonded, stapled or stitched to the body.

The sealing wall may also be a detachable tongue.

The volume of each of the capsules is preferably less than or equal to 20 cm³ or 10 cm³, for example being between 1 and 5 cm³.

Of course, various modifications can be made to the shape of the capsule without departing from the scope of the present invention.

The capsule may comprise several compartments.

The number of compartments contained in the capsule may in particular be other than two, for example being between two and ten.

These compartments make it possible, for example, to contain anhydrous powders that may be completely or partially dissolved when the capsule is opened by a liquid already present in the capsule or outside of the capsule.

These powders may be, for example, salts that increase the ionization of the cosmetic composition or an active agent that is unstable when it is formulated in an aqueous medium. The latter is thus dissolved at the last moment.

When the capsule comprises several compartments, these may be delimited by partitions, which makes it possible to contain compositions separately.

The walls may be made at least partially from a single part with the body of the capsule, for example by moulding.

Optionally, the walls may, for example, have openings that pass through them.

The capsule may advantageously comprise an elastic return member, for example a helical spring, positioned in particular in the bottom of the capsule.

The application member is preferably compressible.

In this case, it may be positioned in the housing so as to be:
in a compressed state when the capsule is sealed by the lid, and
in an uncompressed state by protruding out of the housing when the lid is open.

The application member may also be positioned bearing against the elastic return member, so as to be axially displaceable during the opening of the lid.

Preferably, the application member may be moved between a first position in which it is entirely housed within the housing, and a second position in which it partially protrudes out of the housing, its application surface being located outside of the capsule.

Cosmetic Composition

The cosmetic composition generally comprises at least one active principle.

The active principle is preferably not charged.

The term “charged” is understood to mean any active principle present at least partially in ionic form, the ions of which have an either positive or negative net charge, capable of ensuring their mobility within the composition under the effect of an electric field. Thus, the active agent is directly subjected to the attraction or repulsion of the electrodes.

The composition may comprise a composition for activating an unactivated active principle present within the capsule, for example in freeze-dried form. In this case, the composition may be free of a charged or uncharged active principle. The composition may comprise a solvent having species that are preferably uncharged in order to avoid the competing passage of ions, preferably deionized water.

The activation composition may be contained in a first compartment and the cosmetic composition may be contained in a second compartment.

The compartments may be brought into contact during the opening of the capsule.

Preferably, the activation composition is partially or completely soluble in the cosmetic composition.

Advantageously, the cosmetic composition is selected from care, washing, purifying, exfoliating, desquamating, massage, slimming, makeup, makeup removal, cleansing or bleaching compositions.

More advantageously, the cosmetic composition is in the form of an aqueous solution, an oil, an emulsion, a powder or a gel.

Irrespective of the embodiments considered, the device may exert an action on the skin via iontophoresis and/or electroosmosis.

Another advantage of the invention is to be able to offer the consumer a decontaminated composition.

The cosmetic composition is advantageously free of small-sized ions that reduce the effectiveness of the active agents.

Advantageously, the cosmetic composition is a decontaminated cosmetic composition, in particular that is pasteurized or sterilized. The cosmetic composition advantageously contains no preserving agents whatsoever. To this end, it does not comprise agents defined in Regulation (EC) No 1223/2009 of the European Parliament and of the Council of 30 Nov. 2009 on cosmetic products.

As a variant or in addition, the composition comprises less than 10% by weight of antimicrobial agents such as ethanol, or less than 5% by weight of antimicrobial agents, or even does not contain any antimicrobial agents at all.

The term “decontaminated” should be understood as meaning in particular that the content of bacteria is less than 1000 CFU/g, this threshold being an example of a decontamination threshold.

The term “pasteurized” should be understood as meaning that the content of bacteria of the mesophilic aerobic microorganism type and of the yeast and mould type is in each case less than 1000 CFU/g for cosmetics away from the eyes and in each case less than 100 CFU/g for sensitive products intended for the contours of the eyes and for babies.

These thresholds are determined for example by the standards ISO21149 and ISO16212.

Electrochemical Reaction

Generally, when it is sought to administer an active principle using the device according to the invention, said active principle has the same polarity as the electrode. For example, the compounds containing active principles of positive polarity/charge, such as vitamin A, tocopheryl acetate or other active principles of positive charge/polarity, may be combined with an electrode of positive polarity.

The compounds containing active principles of negative polarity/charge such as retinyl palmitate, tocopherol or mandelic acid may, for their part, be combined with an electrode of negative polarity.

It is possible to treat an area of the body with a capsule, the compartments of which all enable the administration of active principles of the same polarity.

It is also possible that certain compartments enable the administration of an active principle of one polarity and the other compartments allow the administration of an active principle of opposite polarity. In any case, the polarity of the electrodes and of the counterelectrodes is determined by the polarity of the active principles of each compartment.

Method for Using the Capsule

According to another of its aspects, one subject of the invention is a method, in particular a non-therapeutic, cosmetic cleansing method, for extracting compounds from the skin, for example from the epidermis, comprising at least the successive steps consisting in:
putting a capsule as defined above in place on a body area, in particular on the face, and
circulating an electric current within said capsule thus placed in order to enable the extraction of at least one compound present within the skin.

This method may be a method for cleansing the skin, especially that of the face. In this case, the capsule may cover, at least partly, the nose and/or the forehead and/or the cheekbones. The electric current may promote the migration of one or more species from the skin to the capsule, it being intended for these species to be eliminated or to help to convey away, in their migration, one or more compounds to be eliminated.

The compounds extracted from the skin may, for example, be impurities, ions, peptides, proteins, amino acids, polysaccharides, residues of makeup or deposits of dust.

The compounds extracted from the skin may also be residues of a composition previously applied, for example by a mask.

The compounds extracted from the skin may or may not be charged. When these compounds are charged, they may have a polarity opposite to that of the electrode to which they are attracted.

When these compounds are not charged, the extraction thereof may take place by electroosmosis. Thus, the extraction, for example, of positively charged sodium ions may make it possible to create, via an osmotic effect, a flow of water that will entrain uncharged and water-soluble impurities, such as polysaccharides or polar lipids.

Independently of or in combination with the foregoing, one subject of the invention is, according to another of its aspects, a cosmetic method, comprising the steps consisting in:
providing a device as defined above that comprises in particular:
   a cosmetic composition, comprising an active principle, and/or
   a composition for activating an unactivated active principle present within the cosmetic composition,
putting the device in place on a body area, and
circulating an electric current between the skin and the device in place.

The capsules may be heated in order to promote the ionization of the composition contained.

Method for Manufacturing the Device

Another subject of the invention is a method for manufacturing a device according to the invention comprising a step of decontaminating the capsule containing the cosmetic composition (P).

Preferably, the method for manufacturing an aforementioned device comprises the following steps:

(i) providing a non-decontaminated device comprising:
a capsule that delimits a housing,
an application member positioned in the housing and impregnated with a cosmetic composition,
an electric exciter comprising at least two electrodes in permanent contact with the application member and making it possible to circulate an electric current between these two electrodes,

(ii) decontaminating the device, in particular by heating to a decontamination temperature, preferably by gamma ray irradiation or by exposure to ultraviolet or infrared rays or by ethylene oxide treatment.

Preferably, the method comprises a step of decontaminating the capsule. More preferably, the decontamination is a pasteurization or sterilization.

The method for manufacturing the device according to the invention is suitable for reducing the number of bacteria present in the cosmetic composition below a decontamination threshold, in particular a pasteurization or sterilization threshold.

The heating may be carried out for example by microwave heating up to a decontamination temperature.

Advantageously, the method additionally comprises a step of forced cooling of the device, by introduction into a cooling cell.

Generally, the decontamination temperature is greater than or equal to 85° C., preferably between 85° C. and 150° C.

More advantageously, the capsule is closed before the decontamination.

Gripping Member

The device according to the invention advantageously comprises a gripping member.

The gripping member may comprise a compartment in order to house an electric power source that makes it possible to electrically power the electric exciter.

The compartment may be provided with an elastic return member arranged in order to move the electric power source from a first position, in which the electric power source powers the electrodes, to a second position in which the electric power source is disconnected from the electrodes.

Advantageously, the capsule may be detached from the gripping member.

The present invention may be better understood from reading the following detailed description of non-limiting implementation examples of the invention and from examining the appended drawing, in which:

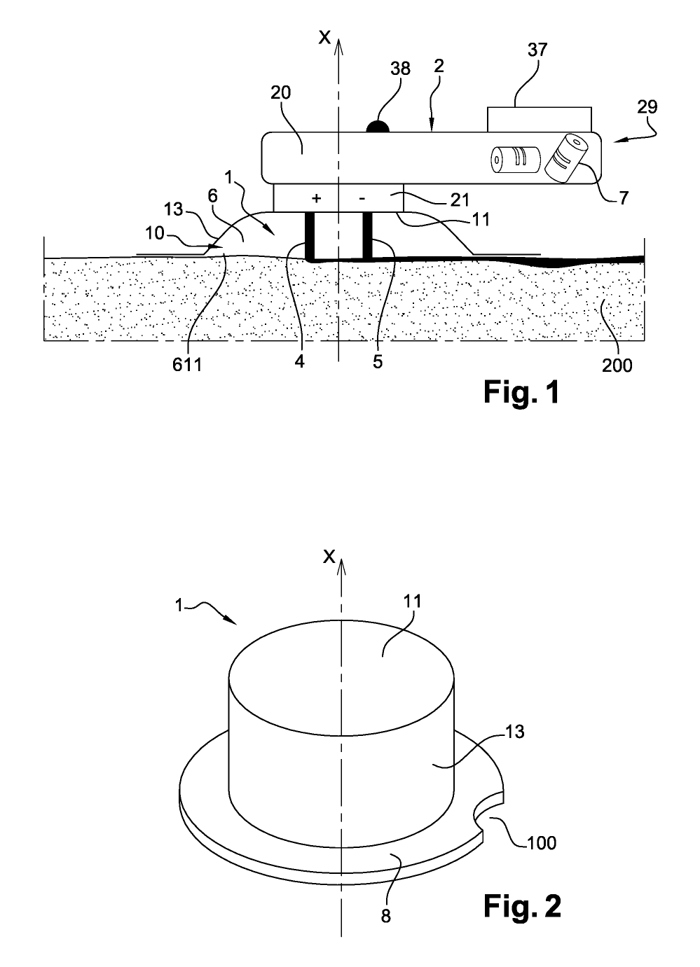

FIG. 1 schematically and partially represents, in axial cross section, an application device in accordance with the invention,

FIG. 2 schematically represents, in perspective, an example of a capsule of the device according to the invention,

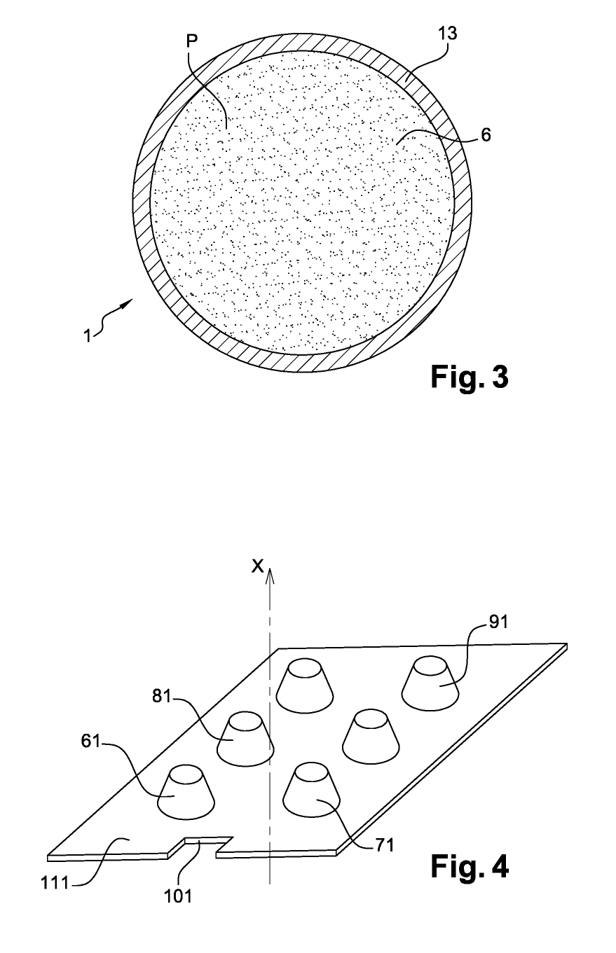

FIG. 3 is a schematic and partial transverse cross-sectional view of a capsule from FIG. 2,

FIG. 4 schematically and partially represents a palette comprising several capsules to be mounted on a gripping member in order to form a device according to the invention.

Represented in FIG. 1 is an application device 1 in accordance with the invention, comprising a capsule 1 and a gripping member 2, the capsule 1 possibly being separated from the gripping member 2. This device is provided in order to apply a composition P.

The capsule 1 comprises a housing 10, of axis X. The housing 10 is delimited by a bottom wall 11 and a side wall 13.

In the example in question, the composition P is a cosmetic or dermatological composition intended to be applied to the skin. As a variant, the composition P may be a hair composition. The composition P is for example a liquid or gel.

The composition P may contain at least one cosmetic or dermatological active agent, in particular at least one compound selected from the following list: metals and alloys thereof, cobalt, barium, chromium, aluminium, silver, copper, titanium, bronze, manganese, metal oxides, iron oxides, in particular ferrite, rare earths, silicates, sulfates, in particular barium sulfate, carbonates, in particular calcium carbonate, non-ferrous compounds, in particular sulfur, magnesium, calcium, boron, potassium, carbon, trace elements, sea salt, rock salts, clay, steatite, algae and planktons and extracts thereof, roots, liquorice, ginger, oily waxes, proteins, hormones, collagens, alums, in particular alum stone, glucose, vitamins, in particular vitamin C, vitamin A, vitamin F, vitamin B, vitamin E, vegetable extracts, glycerin, laponite, surfactants, collagen, acids, in particular salicylic acid, thio acid, caffeine, aromatic essential oils, dyes, antioxidants, free radical scavengers, water-absorbing agents, moisturizers, depigmenting agents, lipid-regulating agents, anti-acne agents, anti-seborrhoeic agents, anti-ageing agents, emollients, antiwrinkle agents, keratolytic agents, anti-inflammatories, refreshing agents, healing agents, vascular protectors, antibacterial agents, antifungal agents, antiperspirants, deodorants, skin conditioners, insensitizing agents, immunomodulators, nourishing agents.

The device comprises a handle 20 of longitudinal axis, for example perpendicular to the axis X.

As a variant, the handle 20 and the capsule 1 may be arranged in order to allow a positioning by snap-fastening, by friction or by bayonet-type locking, of the capsule 1 on a skirt 21 of the handle 20.

The handle 20 and skirt 21 may be produced by moulding as a single part made of plastic.

The application member 3 is housed in the housing 10.

The application member 3 is impregnated with a composition P.

The application member 6 has an outer surface 611 capable of being loaded with composition P and that is used for the application.

In the example considered, the application member 6 comprises a foam. As a variant, the application member 6 may comprise a felt or a sintered product for example.

Before use, the capsule may be sealed by a lid (not represented).

The application number 6 may be configured so that, before opening the lid, the application member 6 is received in the housing 10 by being compressed. As a variant, the application member 6 is received in the housing 8 without being compressed.

The device comprises an electric exciter 29 provided with two electrodes 4 and 5 that may be electrically connected to an electric power source housed in a compartment of the handle 20. These electrodes 4 and 5 are for example rigidly fastened to the handle 20.

The electric power source may comprise, for example, a plurality of batteries 7 arranged in series. As a variant, the electric power source may comprise a dry battery of 4.5 or 9 volts, for example. Of course, the invention is not limited to a particular electric power source, and use may be made of other types of electric batteries or rechargeable batteries or batteries of predefined intensity or else an AC adapter, not represented.

The voltage delivered by the electrodes 4 and 5 is, for example, a continuous voltage, but the electric exciter 29 could comprise an electronic circuit that makes it possible, for example, to deliver a pulsed or alternating current of lower or higher voltage relative to that delivered by the source.

The electric exciter 29 may comprise a switch 37 positioned on an outer face of the gripping member 2, making it possible to control the electric power supply of the electrodes 4 and 5.

The electric exciter 29 may also comprise an indicator light 38 that may light up for example when a voltage is applied to the electrodes 4 and 5.

As a variant, the indicator light 38 may only light up when an electric current of predefined intensity circulates between the electrodes 4 and 5.

As another variant, the indicator light 38 may light up for example with a first colour when the electrodes 4 and 5 are live and light up with a second colour when an electric current circulates between the two electrodes 4 and 5. The indicator light may then be, in this case, a bi-colour light-emitting diode.

In the example in question, the electrodes 4 and 5 are substantially cylindrical and each comprise a terminal portion completely embedded, permanently, in the application member 6, even when this application member is compressed.

Of course, when the electrodes 4 and 5 have a different shape, it being possible for the electrodes 4 and 5 to be flexible or rigid, and metallic or non-metallic, this does not depart from the scope of the present invention.

The electrodes 4 and 5 may also have, where appropriate, a curved shape so as to assist with keeping the application member 6 in the capsule 1.

The electrodes 4 and 5 may or may not be concentric.

The electrodes 30 may be fastened by any means to the rest of the device, in particular by crimping, welding, snap-fastening, over-moulding.

The skirt 21 may optionally be metallic and constitute an electrode.

The application device may be used, for example, in the following manner.

The user removes a lid that covers the application surface 6.

He/she may bring the surface 611 of the application member 6 into contact with the area to be treated, for example the skin.

At the same time as the composition P is applied to the area to be treated, the user may activate the switch 37 in order to electrically power the electrodes 4 and 5.

Since the application member 6 is loaded with composition P, which is electrically conductive, an electric current may circulate between the two electrodes 4 and 5 and in the region of the body in contact with the surface 611 of the application member 6.

The flow of current over the skin may activate the blood microcirculation in particular, which may optionally facilitate the penetration of at least one active agent contained in the composition into the skin.

As a variant, the user may use the device to simply apply the composition to an area to be treated, without generating electric current.

As another variant, the user may, firstly, apply the composition to the area to be treated using the device without generating electric current, then, secondly, once the composition has been applied, generating an electric current on the skin while massaging the treated area for example.

The user may also alternate treatments with application of an electric current and treatments without.

The electric current may also be applied periodically, for example by manipulating the switch 37 so as to cause the flow of electric current intermittently.

In the example from FIG. 2, the capsule 1 is produced by moulding material as a single part.

In the example illustrated, the housing 10 is produced with a flange 8 at one end and has, for example, a shape that is generally rotationally symmetrical about an axis X.

The flange 8 may be provided with a mistake-proofing device 100 that is for example in the form of a recessed or protruding relief, for example a notch.

The capsule comprises a bottom 11 and a side wall 13.

The capsule 1 comprises for example a film that is heat-sealed to the flange 8 so as to seal it in an airtight manner.

FIG. 3 shows the composition P impregnated on the substrate 6.

FIG. 4 shows a palette 111 of capsules intended to equip a device according to the invention. The capsules may be identical or different. For example, they may contain complementary products, such as care products and makeup products. The palette 11 may be provided with a notch 101 in order to facilitate the gripping thereof.

The user may for example use the palette as follows:
he/she prepares his/her face 200,
he/she places a capsule 61 comprising an exfoliating composition on the gripping member 2,
he/she places the capsule 61 on his/her face 200,
he/she actuates the device according to the invention for 2 minutes,
he/she detaches the consumed capsule 61 and replaces it with a care capsule 71,
he/she places the capsule 71 on his/her face 200,
he/she actuates the device according to the invention for 2 minutes,
he/she detaches the consumed capsule 71 and replaces it with a capsule 81 for making up the cheeks and/or with a capsule 91 for making up the eye contour area,
he/she actuates the device according to the invention for 2 minutes.

The invention is not limited to the devices described in the examples. In particular, the capsule may have a different structure and contain another application member. The composition may be different. The electric exciter may comprise other elements.

Throughout the description, including the claims, the term “comprising a” should be understood as being synonymous with “comprising at least one”, unless otherwise specified.

1. Device for applying a cosmetic composition (P), comprising:
a capsule (1) that delimits a housing (10),
an application member (6) positioned in the housing (10) and impregnated with a cosmetic composition (P),
an electric exciter (29) comprising at least two electrodes (4, 5) in contact with the application member (6) and making it possible to circulate an electric current between these two electrodes (4, 5),
the capsule (1) being made of soft material.

2. Device according to claim 1, the capsule (1) being removable and disposable.

3. Device according to either one of the preceding claims, the capsule (1) comprising a multilayer material.

4. Device according to any one of the preceding claims, the capsule (1) being obtained by means of a beam of metal ions.

5. Device according to any one of the preceding claims, the capsule (1) being closed by a lid.

6. Device according to any one of the preceding claims, the application member (6) being compressible.

7. Device according to any one of the preceding claims, the application member (6) being porous or fibrous.

8. Device according to any one of the preceding claims, the application member (6) being selected from a nonwoven, a felt, a paper, a foam, a sponge, a sintered product, a porous ceramic or a biocellulose substrate.

9. Device according to any one of the preceding claims, designed so that the current is applied continuously or sequentially.

10. Device according to any one of the preceding claims, the capsule (1) comprising several compartments.

11. Capsule intended to be fitted on a device according to any one of the preceding claims.

12. Cosmetic method comprising the steps consisting in:
providing a device as defined in any one of claims 1 to 10,
putting the device in place on a body area, and
circulating an electric current between the skin and the device in place.

13. Method for manufacturing a device as defined according to any one of claims 1 to 10, comprising a step of decontaminating the capsule (1).
